(19) United States
(12) Patent Application Publication
(10) Pub. No.: US 2026/0091208 A1
(43) Pub. Date: Apr. 2, 2026

(54) EXPANDABLE DRUG DELIVERY DEVICE WITH ENHANCED NEEDLE PENETRATION PRESSURE

(71) Applicant: Alma Therapeutics Ltd., Kfar Sava (IL)

(72) Inventors: Yossi Gross, Mazor (IL); Oz Cabiri, Hod HaSharon (IL)

(73) Assignee: Alma Therapeutics Ltd., Kfar Sava (IL)

(21) Appl. No.: 18/903,136

(22) Filed: Oct. 1, 2024

(51) Int. Cl. A61M 31/00; A61M 39/22

(52) U.S. Cl. CPC A61M 31/002; A61M 39/22

(57) ABSTRACT

A drug delivery device includes an ingestible pill including an enteric coating that covers a patch which defines therein a chamber, the patch being constructed to allow entry of liquid into the chamber. The patch includes at least one raised pouch that protrudes outwardly from the patch and which is in fluid communication with the chamber. The at least one raised pouch includes at least one drug-delivery needle. A gas-generating substance is disposed in the chamber, The gas-generating substance is configured to release gas after the liquid has entered the chamber, the gas being flowable into the at least one raised pouch.

### FIELD OF THE INVENTION

The present invention relates generally to drug delivery needles, and particularly to a drug delivery device that expands to drive a needle into tissue with enhanced needle penetration pressure.

### BACKGROUND OF THE INVENTION

Drug delivery into mucosal tissue presents many challenges. In many places in the body, mucosal tissue includes three basic layers: the epithelium (the outermost layer), lamina propria, and the deeper sub-mucosal tissue. These three layers may be supported by annular muscles or tissue (such as the serosa). The lamina propria is rich in macrophages and lymphoid cells, and forms part of the barrier that protects internal tissues from external pathogenic microorganisms.

The ability for a drug to pass the epithelium and reach the lamina propria and the sub-mucosal tissue is a challenge. In particular, drug delivery by needle penetration into the mucosa has many challenges. For example, the mucosa in the gastrointestinal (GI) tract is covered with a continuously renewed viscous fluid which prevents needle penetration at many different angles of the needle tip with respect to the tissue. A major problem is trying to penetrate tissue with short-length needles. The short length is required to avoid penetrating abdominal tissue which must not be penetrated. However, the short length often fails to penetrate sufficiently to deliver the drug to the tissue. Another difficulty is the resilience to the mucosa to needle penetration; there is generally no counterforce to the needle penetration. The force of the needle tip trying to penetrate the mucosa causes the mucosa to be pushed in the direction of the force without the needle tip succeeding to penetrate the mucosa.

### SUMMARY

The present invention seeks to provide an expandable drug delivery device that expands to drive a needle into tissue with enhanced needle penetration pressure, as described in detail below.

In a non-limiting embodiment of the invention, the drug-delivery needle is located on a pouch that becomes filled with gas generated by a gas-generating substance in the device. The pouch is pushed by the gas towards the tissue so that the needle penetrates the tissue. The area upon which the pushing force of the gas acts is concentrated to a much smaller area (the distal area of the pouch with the needles; the pouch being the area raised above the surface of the balloon, patch or other pushing member) which greatly increases the penetration pressure of the needles. As a result the needles of the invention successfully penetrate the tissue to the required depth, limited by the needle length as required.

There is provided in accordance with a non-limiting embodiment of the invention a drug delivery a drug delivery device including an ingestible pill including an enteric coating that covers a patch which defines therein a chamber, the patch being constructed to allow entry of liquid into the chamber, wherein the patch includes at least one raised pouch that protrudes outwardly from the patch and which is in fluid communication with the chamber, the at least one raised pouch including at least one drug-delivery needle, and a gas-generating substance disposed in the chamber, the gas-generating substance being configured to release gas after the liquid has entered the chamber, the gas being flowable into the at least one raised pouch.

In accordance with a non-limiting embodiment of the invention a pushing force of the gas generated by the gas-generating substance applied to the at least one drug-delivery needle is concentrated on a distal portion of the at least one raised pouch where the at least one drug-delivery needle is located.

In accordance with a non-limiting embodiment of the invention the at least one raised pouch is formed with an entry port for gas flow therein.

### BRIEF DESCRIPTION OF DRAWINGS

The present invention will be understood and appreciated more fully from the following detailed description, taken in conjunction with the drawings in which:

FIG. 1 is a simplified perspective illustration of a drug delivery device, in accordance with a non-limiting embodiment of the invention.

FIGS. 2A and 2B are side-view and planar-view illustrations, respectively, of a prior art patch expanding and pushing needles into a tissue, wherein FIG. 2B shows the area of the patch which is pressed against the tissue.

FIGS. 3A and 3B are side-view and planar-view illustrations, respectively, of the patch of the present invention with raised pouches that expand and push needles into the tissue, wherein FIG. 3B shows the area of the raised pouch which is pressed against the tissue.

FIG. 4 is a simplified perspective illustration of the raised pouch with the drug delivery needles, in accordance with a non-limiting embodiment of the invention.

FIG. 5 is a cutaway illustration of the drug delivery device showing paths for gas to enter the raised pouches with the needles. It is noted that the pouches may have a very small area with a small amount of gas entering therein. The invention is not limited to this, and the pouch can be rigid or partially rigid, and the pouch can be made of a material that dissolves. The pouch can be created in many ways. For example, folds or blisters may be created in the material of the base, or the pouch may be made from a material that locally expands when exposed to liquid, or they be made by vacuum forming. The pouches afterwards become filled with the gas generated by the gas-generating substance in the device.

FIG. 6 is a simplified perspective illustration of the drug delivery device configured as a balloon, in accordance with a non-limiting embodiment of the invention.

FIG. 7A is a simplified perspective illustration of the drug delivery device configured as a toroid, in accordance with a non-limiting embodiment of the invention.

FIGS. 7B and 7C are simplified perspective illustrations of the drug delivery device configured as a patch, with and without a central hole, in accordance with a non-limiting embodiment of the invention.

FIGS. 8A-8D are simplified top view, sectional view, side view and perspective views, respectively, of an array of pouches with needles, in accordance with a non-limiting embodiment of the invention, with FIG. 8B taken along lines B-B in FIG. 8A.

FIGS. 9A and 9B are simplified side view illustrations of the needles of FIGS. 8A-8D, respectively, before and after penetrating tissue.

### DETAILED DESCRIPTION

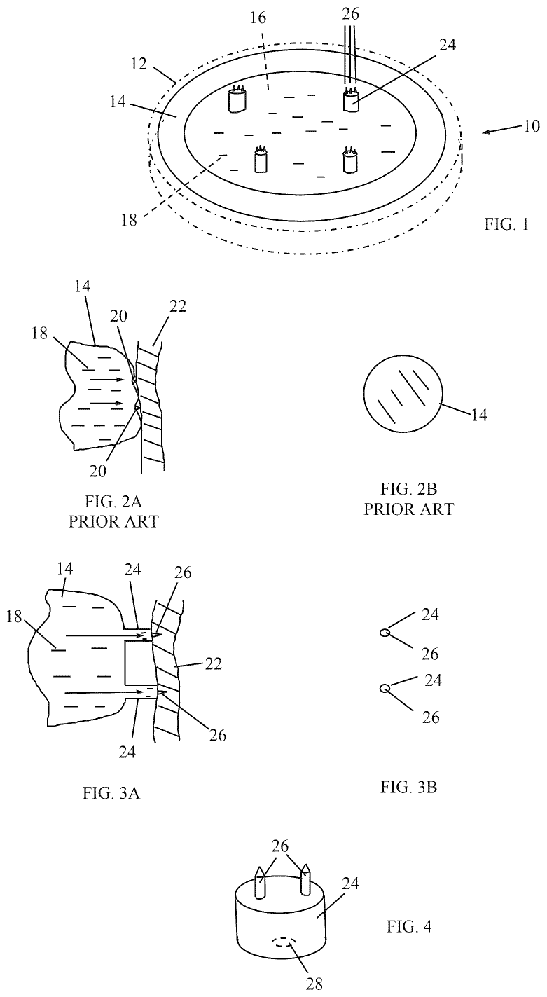

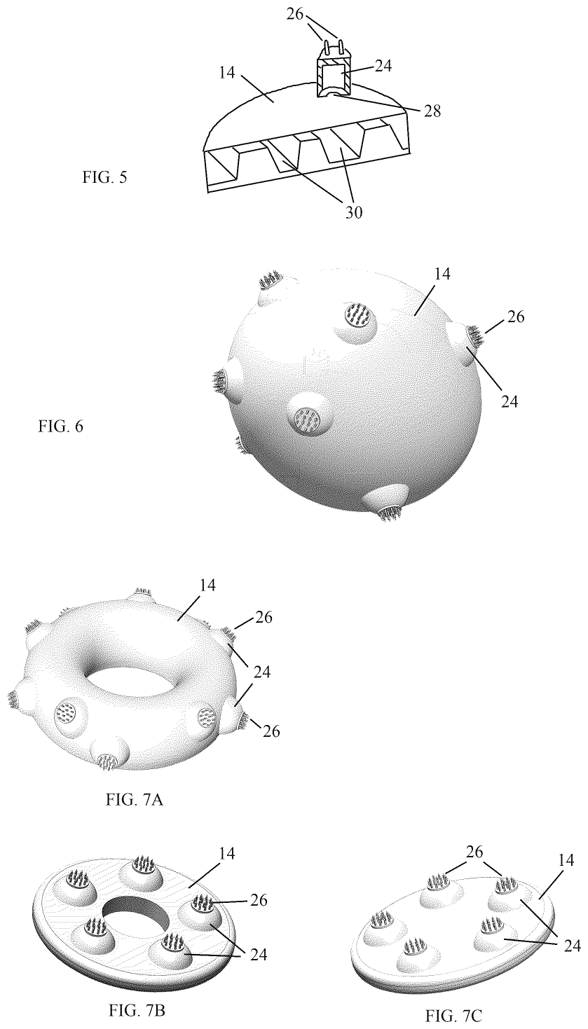

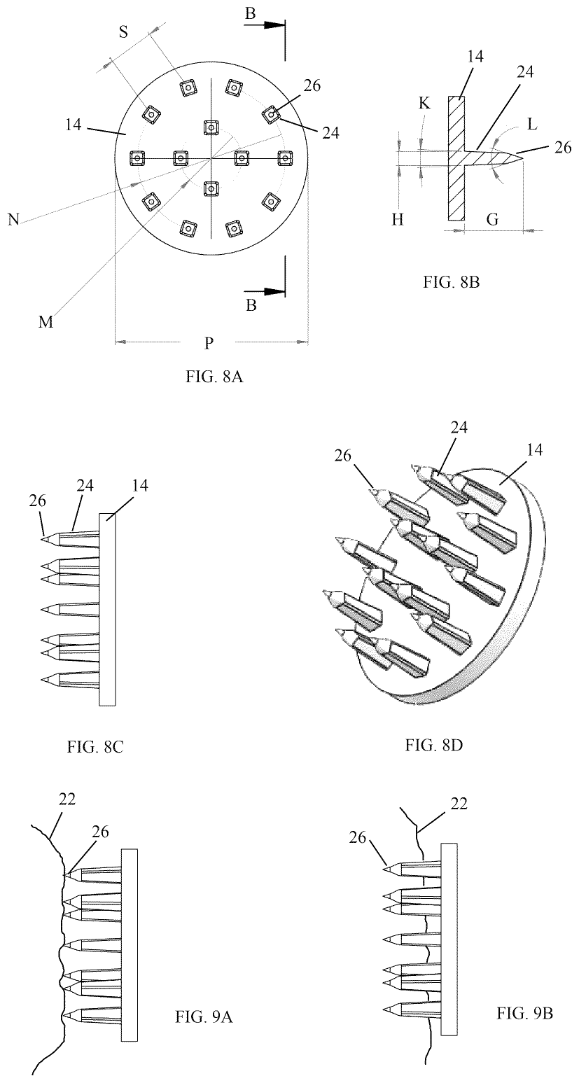

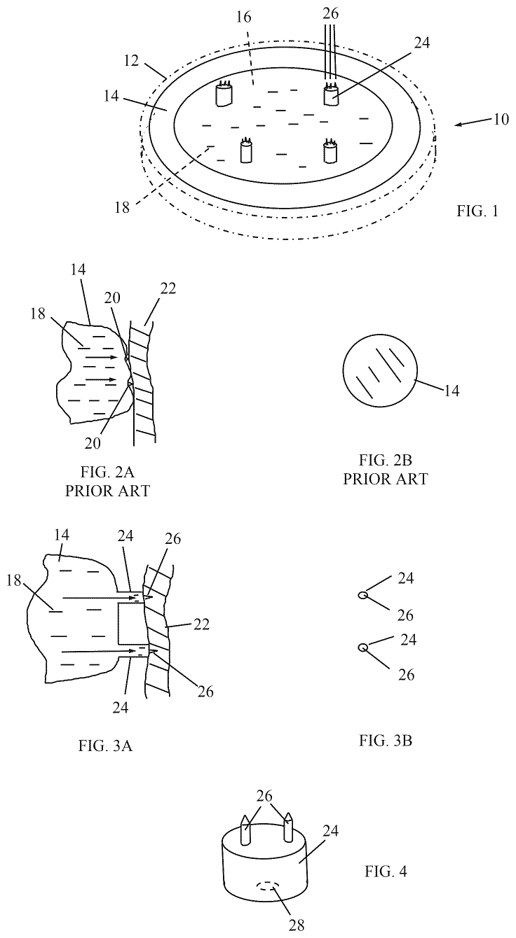

Reference is now made to FIG. 1, which illustrate a drug delivery device 10, in accordance with a non-limiting embodiment of the invention.

In a non-limiting embodiment of the invention, portions of the drug delivery device 10 may be similar to the drug delivery devices described in PCT Patent Application WO 2020/035857, and as such, may be in the form of an ingestible pill that has an enteric coating 12, which covers a patch 14 (or balloon, the terms being used interchangeably) which defines therein a chamber 16. Patch 14 is constructed to allow entry of liquid, such as from a gastrointestinal (GI) tract after ingestion of the pill, into chamber 16 (e.g., without limitation, patch 14 may be porous or may have apertures or may let the liquid soak through the thickness of the patch to seep into chamber 16). A gas-generating substance 18 (e.g., without limitation, sodium bicarbonate or other gas generating substances) is disposed in chamber 16. Liquid that has entered chamber 16 contacts gas-generating substance 18, and causes gas-generating substance 18 to release gas (such as by means of the liquid causing a chemical reaction with gas-generating substance 18). The gas causes chamber 16 to expand, thereby pushing the outer contour of patch 14 against tissue in the GI tract.

Reference is now made to FIGS. 2A and 2B. In the prior art (such as in PCT Patent Application WO 2020/035857), the patch 14 includes needles 20, which upon expansion of patch 14, are pushed into a tissue 22. Needles 20 are configured to deliver a drug into tissue 22. However, as seen in FIG. 2A, the expansion of patch 14 may cause areas of patch 14 near the needles 20 to be pushed as well against tissue 22. The portions of patch 14 which push against tissue 22 can tend to push the tissue 22 away from needles 20. As a result, needles 20 cannot penetrate tissue 22 to deliver the drug they are supposed to deliver. FIG. 2B shows the area of the patch 14 which is pressed against the tissue 22.

Reference is now made to FIG. 1 and to FIGS. 3A and 3B. In accordance with a non-limiting embodiment of the invention, patch 14 includes one or more raised pouches 24, which protrude outwardly from patch 14 and which are formed as bubbles, blisters, blebs, and the like (all of these terms being used interchangeably). Each raised pouch 24 is in fluid communication with chamber 16, so that any gas generated by gas-generating substance 18 enters the raised pouch 24. One or more needles 26 are positioned on each raised pouch 24. As used throughout the description and claims, the term needle encompasses any sharp penetration member. Here, too, needles 26 are configured to deliver a drug into tissue 22. Without limitation, the drug may either flow through the needle or the needle may be coated with the drug or the needle tip may be made of the drug.

As seen in FIG. 3A, gas generated by gas-generating substance 18 enters the raised pouch 24. Contrary to the prior art, in the invention, the raised pouches 24 with needles 26 are pushed into the tissue 22. The area upon which the pushing force of the gas acts is concentrated to a much smaller area (the distal area of the pouch with the needles) compared with the prior art, as seen by comparing FIG. 3B of the invention with FIG. 2B of the prior art. Since pressure is force divided by area, this means that for the same gas expansion force, the pressure at the needle 26 of the invention is much greater than the pressure at needle 20 of the prior art. As a result the needles 26 of the invention successfully penetrate tissue 22 to the required depth (e.g., without limitation, into the submucosa layer, penetrating not more than 3 mm) and successfully deliver the drug to the tissue 22.

Reference is now made to FIG. 4, which illustrates the raised pouch 24 with the drug delivery needles 26, in accordance with a non-limiting embodiment of the invention. As mentioned above, raised pouch 24 is in fluid communication with chamber 16 (FIG. 1), so that any gas generated by gas-generating substance 18 enters the raised pouch 24 through an entry port 28 (FIG. 4), Entry port 28 may be a simple hole or even a valve, such as a one-way valve which will keep the gas in the pouch against the counterforce of the tissue. In all embodiments of the invention, the raised pouches 24 may be made by vacuum forming or other methods, and they afterwards become filled with the gas generated by the gas-generating substance in the device.

Reference is now made to FIG. 5. It is seen that patch 14 may be formed with one or more partitions 30 that define paths for the gas to enter the entry port 28 into the raised pouch 24.

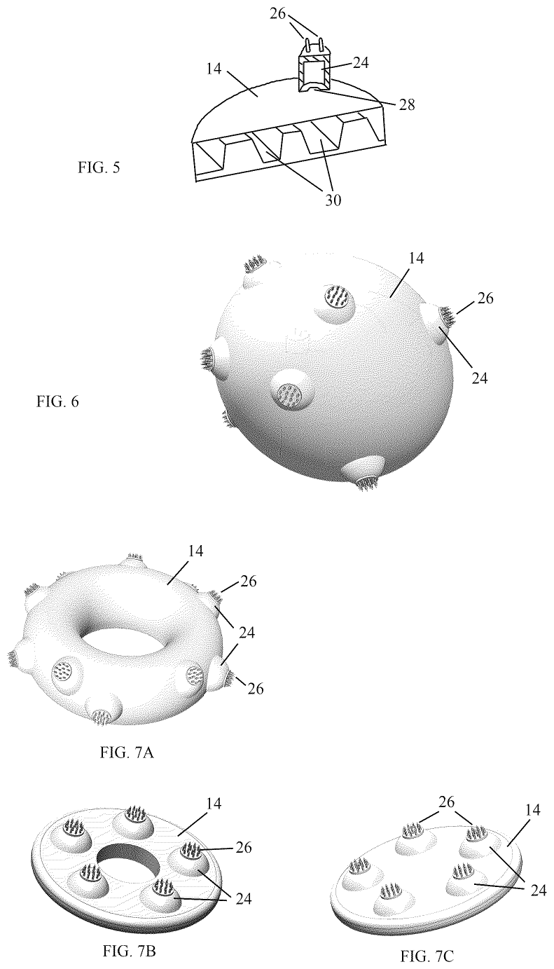

The drug delivery device may have any shape and size. Reference is now made to FIG. 6, which shows one example of the drug delivery device configured as a balloon.

Reference is now made to FIG. 7A, which shows another example of the drug delivery device configured as a toroid. FIGS. 7B and 7C illustrate the drug delivery device configured as a patch, with and without a central hole.

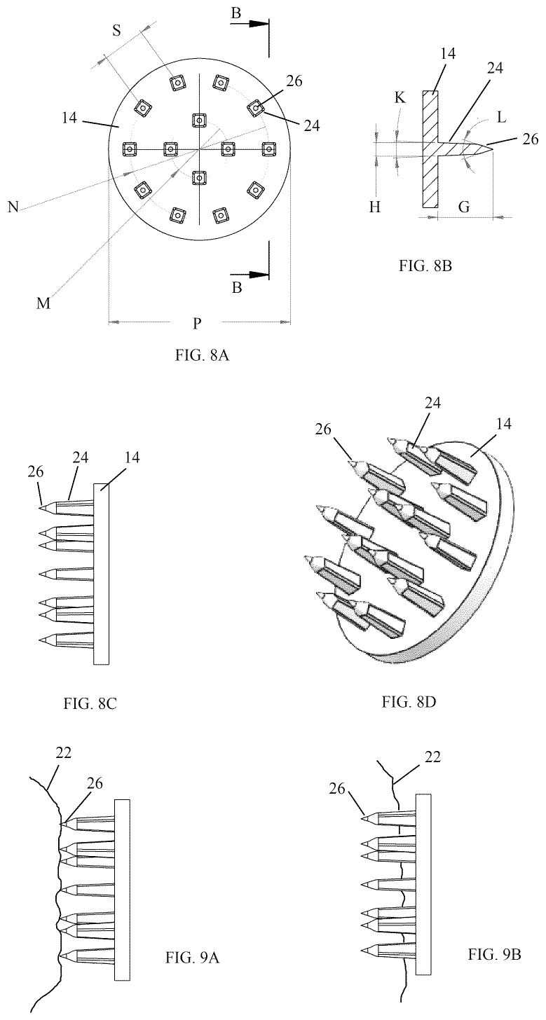

Reference is now made to FIGS. 8A-8D, which illustrate another array of pouches 24 with needles 26, in accordance with a non-limiting embodiment of the invention. Without limitation, pouches 24 and needles 26 may be arranged on patch 14 in an inner circular array (diameter M, without limitation, 1-3 mm) and an outer circular array (diameter N, without limitation, 4-8 mm). The circumferential spacing S between the needles on the outer circular array may be, without limitation, 1-4 mm. The diameter P of the patch 14 may be, without limitation, 4-12 mm. The thickness H of the pouch 24 may be, without limitation, 0.3-1 mm. The pouch 24 may be tapered slightly at an angle K, without limitation, 3-6°. The needle 26 may be tapered at an angle L, without limitation, 25-45°.

Reference is now made to FIGS. 9A and 9B. Once again, the needles 26 of the invention successfully penetrate tissue 22 to the required depth (e.g., without limitation, into the submucosa layer, penetrating not more than 3 mm) and successfully deliver the drug to the tissue 22.

1. A drug delivery device comprising:
an ingestible pill comprising an enteric coating that covers a patch which defines therein a chamber, said patch being constructed to allow entry of liquid into said chamber;
wherein said patch comprises at least one raised pouch that protrudes outwardly from said patch and which is in fluid communication with said chamber, said at least one raised pouch comprising at least one drug-delivery needle; and
a gas-generating substance disposed in said chamber, said gas-generating substance being configured to release gas after the liquid has entered said chamber, the gas being flowable into said at least one raised pouch.

2. The drug delivery device according to claim 1, wherein a pushing force of the gas generated by said gas-generating substance applied to said at least one drug-delivery needle is concentrated on a concentrated area of a distal portion of said at least one raised pouch where said at least one drug-delivery needle is located, said concentrated area of the distal portion of said at least one raised pouch being less than a surface area of said pouch.

3. The drug delivery device according to claim 1, wherein said at least one raised pouch is formed with an entry port for gas flow therein.

4. The drug delivery device according to claim 3, wherein said entry port is a hole.

5. The drug delivery device according to claim 3, wherein said entry port comprises a valve.

6. The drug delivery device according to claim 1, wherein said patch is formed with one or more partitions that define paths for the gas to enter said at least one raised pouch.

7. The drug delivery device according to claim 1, wherein said drug delivery device is configured as a balloon.

8. The drug delivery device according to claim 1, wherein said drug delivery device is configured as a toroid.

9. The drug delivery device according to claim 1, wherein said at least one raised pouch is formed as a bubble that protrudes from an outer surface of said patch.

10. The drug delivery device according to claim 1, wherein said at least one raised pouch is formed as a blister that protrudes from an outer surface of said patch.

11. The drug delivery device according to claim 1, wherein said at least one raised pouch is formed as a bleb that protrudes from an outer surface of said patch.
